(19) United States
(12) Patent Application Publication
(10) Pub. No.: US 2021/0151199 A1
(43) Pub. Date: May 20, 2021

(54) TOOL AND METHODS OF USE FOR SYNCHRONOUS AND ASYNCHRONOUS COMMUNICATION BETWEEN PATIENTS AND HEALTHCARE PROVIDERS

(71) Applicant: Telemedicine Provider Services, LLC, Boulder, CO (US)

(72) Inventors: Kevin McGarvey, Boulder, CO (US); Jason Sperling, Boulder, CO (US); Tom Piekos, Lafayette, CO (US); David Carroll, Corvallis, OR (US); Michael Hellein, Barre, VT (US)

(21) Appl. No.: 16/952,720

(22) Filed: Nov. 19, 2020

Related U.S. Application Data
(60) Provisional application No. 62/937,575, filed on Nov. 19, 2019.
(60) Provisional application No. 63/007,222, filed on Apr. 8, 2020.

(51) Int. Cl. G16H 80/00; G16H 10/60; H04L 29/06

(52) U.S. Cl. CPC G16H 80/00; H04L 65/4023; G16H 10/60

(57) ABSTRACT

A tool for synchronous and asynchronous communication between a patient, such as a pandemic patient, a plurality of healthcare providers, one or more EMR/EHR systems, and one or more third-party service provider systems includes a server operating one or more processors in communication with patient, healthcare provider, third-party service provider, and channel databases, along with a management engine executing a database management module, a rule module, and a GUI module configured to display a GUI having a plurality of preconfigured, interactive screens to users operating at least one healthcare provider terminal and a patient terminal. The management engine may send and receive synchronous and asynchronous communications between the patient, the healthcare providers, the EMR/HER systems, and the third-party service provider systems via a dedicated patient channel and display the communications upon the GUI screens, generating a healthcare transcript on the continuing patient channel. Other embodiments are disclosed.

### REFERENCE TO PENDING PRIOR PATENT APPLICATION

This application claims the benefit under 35 U.S.C. 119 (e) of (1) U.S. Provisional Patent Application No. 63/007,222, filed Apr. 8, 2020 by Kevin McGarvey, et al., for “TOOL AND METHODS OF USE FOR SYNCHRONOUS AND ASYNCHRONOUS COMMUNICATION BETWEEN PANDEMIC PATIENTS, HEALTHCARE PROVIDERS, AND THIRD-PARTY SERVICE PROVIDERS,” and (2) U.S. Provisional Patent Application No. 62/937,575, filed Nov. 19, 2019 by Kevin McGarvey, et al., for “TOOL AND METHODS OF USE FOR SYNCHRONOUS AND ASYNCHRONOUS COMMUNICATION BETWEEN PATIENTS AND HEALTHCARE PROVIDERS,” which patent applications are hereby incorporated herein by reference.

### BACKGROUND

Existing communication systems for patient inquiries to their doctor or medical or healthcare provider's offices suffer from a number of technical limitations. Telephone communication, perhaps the most common tool currently used for communication between patients and their healthcare providers, requires synchronous communication in discrete, but real-time, interactions. Email, text, and chat capabilities, as well as existing patient portals implemented by many provider groups, offer asynchronous communication but lack flexibility in effectively executing common tasks that are germane to the provision of effective medical care, including medical screening, monitoring, triage, escalation, integration of communication with or into virtual tele-health visits, and integration with external third-party applications and/or systems for providing, for example, grocery, supply, and/or pharmaceutical delivery, patient transportation, and emergency care. In addition, medical or healthcare provider inquiries to referral or consultative medical partners suffer from similar technical limitations.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a healthcare communication tool for synchronous and asynchronous communication between a patient and a plurality of healthcare providers of a healthcare provider group. The communication tool may be used to treat pandemic patients such as, for example, COVID-19 patients. The communication tool may include a (1) a communications operations server operating one or more processors in communication with a patient database, a healthcare provider database, a third-party service provider database, and a channel database; and (2) a management engine running on the communications operations server, the management engine executing a database management module, a rule module, and a graphical user interface (GUI) module configured to display a GUI having a plurality of preconfigured, interactive screens to users operating at least one healthcare provider terminal operatively coupled with a plurality of provider tele-health tools and a patient terminal operatively coupled with a plurality of patient tele-health tools, the management engine configured for: (a) initiating, via a patient channel dedicated to the patient, a tele-health visit between the patient and at least one of the plurality of the healthcare providers; (b) asynchronously exchanging, via the patient channel and using the plurality of the patient and the provider tele-health tools, information relating to one or more of patient screening, monitoring, diagnosis, treatment, and triage; (c) reporting to one or more electronic medical record (EMR) systems, the information relating to the one or more of the patient screening, monitoring, diagnosis, treatment, and triage; and (d) requesting, from one or more third-party service provider systems, one or more of an emergency medical services dispatch, a food delivery to the patient, a supply delivery to the patient, a pharmaceutical delivery to the patient, and a non-emergency transportation service for the patient.

Another embodiment provides a healthcare communication tool for synchronous and asynchronous communication between a patient and a plurality of healthcare providers of a healthcare provider group. The tool may include (1) a communications operations server operating one or more processors in communication with a patient database, a provider database, a third-party service provider database, and a channel database; and (2) a management engine running on the communications operations server, the management engine executing a database management module, a rule module, and a graphical user interface (GUI) module configured to display a GUI having a plurality of preconfigured, interactive screens to users operating at least one healthcare provider terminal and a patient terminal, the management engine: (a) receiving, via a patient channel dedicated to the patient and the plurality of the healthcare providers of the healthcare provider group, a healthcare request from the patient operating the patient terminal; (b) displaying, within the patient channel and upon one of the preconfigured, interactive screens of the GUI at the at least one healthcare provider terminal and the patient terminal, the healthcare request; (c) receiving, via the patient channel, a healthcare response from at least one of the plurality of the healthcare providers operating the at least one healthcare provider terminal; and (d) displaying, within the patient channel and upon another one of the preconfigured, interactive screens of the GUI at the at least one healthcare provider terminal and the patient terminal, the healthcare response.

In another embodiment, the management engine may further be configured for (1) asynchronously receiving, via the patient channel, additional healthcare responses from the patient operating the patient terminal and the at least one of the plurality of the healthcare providers operating the at least one healthcare provider terminal; and (2) displaying, within the patient channel and upon another one of the preconfigured, interactive screens of the GUI at the at least one healthcare provider terminal and the patient terminal, the additional healthcare responses to form a continuous patient-channel transcript within the patient channel.

In yet another embodiment, the management engine may further be configured for (1) receiving, via the patient channel, a tele-health visit initiation from the at least one of the plurality of the healthcare providers operating the at least one healthcare provider terminal; (2) receiving, via the patient channel, a tele-health visit acceptance from the patient operating the patient terminal; and (3) initiating, via the patient channel and upon another one of the preconfigured, interactive screens of the GUI at the at least one healthcare provider terminal and the patient terminal, a virtual tele-health visit.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 provides a block diagram depicting an illustrative environment and device ecosystem within and through which a synchronous and asynchronous healthcare communication tool and associated techniques may be implemented according to some embodiments;

FIG. 2 provides a functional block diagram depicting illustrative communications software of the communication tool of FIG. 1 according to some embodiments;

FIGS. 3A-3K illustrate numerous exemplary preconfigured and interactive screens presented at patient and healthcare provider user terminals in implementing the functionality of embodiments of the communication tool and the software of FIGS. 1-2; and

FIG. 4 provides a flowchart depicting an exemplary method of treating a quarantined pandemic patient (e.g., a COVID-19 patient) using the communication tool of FIGS. 1-2 and 3A-3K.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

Various embodiments of the systems and methods described herein generally relate to facilitating secure, web-based, asynchronous and synchronous communication between patients and their healthcare providers via continuous, patient-provider group “channels” dedicated to each patient. Embodiments of the disclosed communication tool are accessible via remote mobile and/or desktop platforms and utilize the patient channels through which patients may communicate asynchronously and synchronously with one or more healthcare provider groups, and with one or more of the individual providers fulfilling various roles within each of those healthcare provider groups (e.g., a doctor, a nurse, a billing professional, an administrative or scheduling professional) via the patient-provider group dedicated channels.

Embodiments of the disclosed communication tool provide the ability for one or more healthcare providers included in a patient's care team to receive notifications regarding inbound patient inquiries for triage and for one or more providers to actively participate in and/or passively monitor communication between the patient and members of the patient's care team. The role of each provider participating on a patient's thread or “channel” may shift over time, enabling escalation to different members of the patient's care team as necessary to provide the best care.

Embodiments of the communication tool may also integrate with other healthcare applications and electronic systems such as, for example, electronic medical record (EMR) systems and/or electronic healthcare record (EHR) systems to port communication snippets, documents, and/or other relevant communications into the patient's ongoing, electronic healthcare record. With video and audio conferencing tools, the system allows for written and documentary communication to seamlessly transition into a virtual tele-health visit between the relevant healthcare provider(s) and the patient. In use, embodiments of the communication tool enable a diverse team of healthcare professionals and support staff to better communicate and more effectively care for the patient to improve clinical outcomes and reduce wasteful, fragmented, episodic care.

Embodiments of the communication tool may additionally integrate with or access third-party service provider applications and/or systems to request, procure, or provide ancillary services for or to the patient, as deemed necessary by members of the patient's care team. Exemplary third-party service provider applications and/or systems may include, for example, systems for patient transportation via a medically secure vehicle or through a non-emergency ride-share applications such as Uber or Lyft, for delivery of over-the-counter and/or prescription pharmaceuticals, for the dispatch/provision of emergency medical services (EMS) and/or emergency response agency services, for the delivery of groceries and/or other household or medical goods or items (e.g., disinfectant wipes, facial masks, toilet or bath tissue, facial tissue, paper towels, etc.), for providing notifications to the patient's friends and/or family through email, phone, and/or social media platforms, and/or for the monitoring, reporting, and/or tracking of disease spread and progression via systems affiliated with local, regional, state, and/or federal organizations such as, for example, the centers for disease control (CDCs). In this regard, embodiments of the communication tool provide an ideal technological platform to deliver telemedicine services to socially isolated or distanced pandemic patients, including COVID-19 patients.

In one embodiment, the communication tool described herein may be implemented within the larger structure of a referral-based care network platform that connects patients with all of the stakeholders invested in their optimal care, including any number of healthcare providers, non-medical-professional caregivers, health advocates, family members, and/or friends, as discussed in U.S. patent application Ser. No. 16/509,683, filed Jul. 12, 2019, entitled “TELE-HEALTH NETWORKING, INTERACTION, AND CARE MATCHING TOOL AND METHOD OF USE,” which is hereby incorporated herein in full. While embodiments of the communication tool disclosed herein detail communications between the patient and the healthcare provider groups and associated healthcare providers that form the patient's care team within a larger care network, the communication tool may be used to facilitate asynchronous and synchronous web-based communication between the patient and the patient's healthcare provider(s), as well as any other appropriate stakeholders that are relevant to the patient's care.

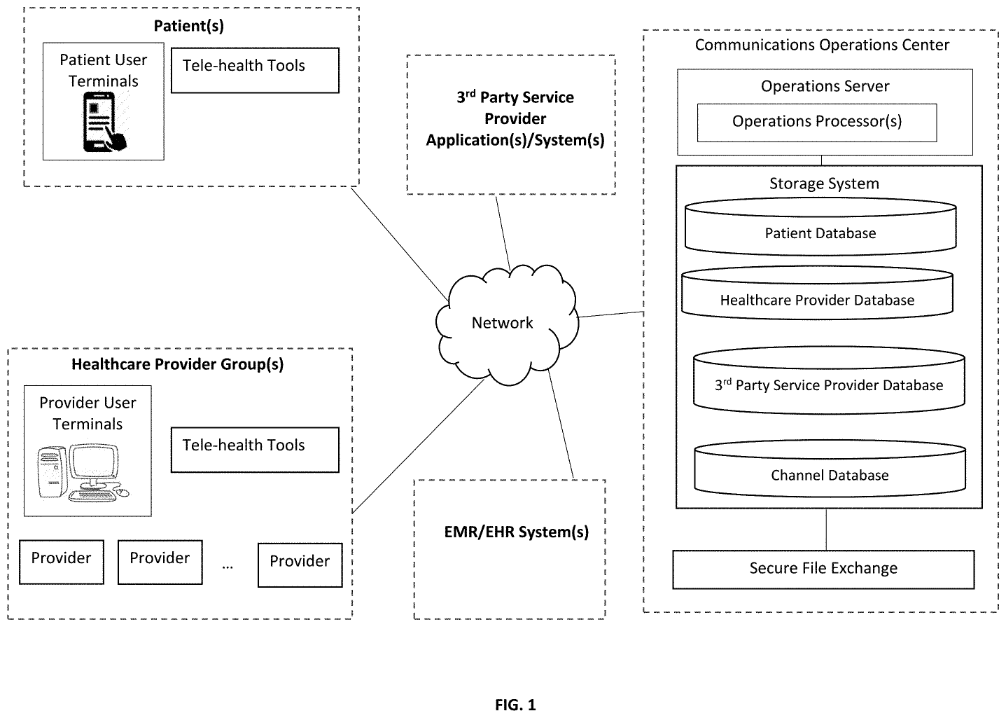

Turning to the figures, FIG. 1 provides a block diagram depicting an illustrative environment and device ecosystem within and through which the communication tool and associated techniques described herein may be implemented according to some embodiments. As shown in FIG. 1, a patient may be communicatively connected with one or more healthcare provider groups, each including a number of individual healthcare providers, via a network of secure communication channels. The healthcare groups may include any appropriate groups such as, for example, a primary care physician group, a hospital group, and/or an emergency medical service (EMS) or emergency care group.

In some embodiments, each of the patients and the healthcare providers of each provider group may operate at least one network-enabled user terminal configured to securely compile and transmit information to and from other system components. Each of the patient and the provider user terminals may be any appropriate network-enabled mobile or desktop user device such as, for example, a desktop computer, laptop computer, tablet computer, smartphone, or the like. Each of the user terminals may incorporate or access a transmission system that provides network communication abilities. An exemplary transmission system may include one or more analog switches, servers, IP gateways, PBX systems, etc. For example, in some embodiments, the transmission system of each user terminal may be operable to provide communications through the network, which may include, for example, the Internet. Additionally or alternatively, the network may include wireless cellular networks or the like.

In addition, the user terminals may each be equipped with or operatively coupled to a variety of HIPAA-compliant, remote collaboration, tele-health tools as appropriate and/or desired. The tele-health tools may provide the modalities for telepresence healthcare interactions and a means of providing data to and communicating with the communication tool components. Such tele-health tools may include, for example, secure chat and messaging tools, video conferencing tools, voice-over Internet technologies, and/or file sharing exchanges.

A communications operations center may be communicatively coupled with the user terminals operating at the patient and the healthcare provider group(s), as well as with one or more EMR/EHR systems and one or more external, third-party service provider applications or systems. In one embodiment, the one or more third-party service provider applications or systems may include, by way of limited example, patient transportation systems (e.g., ambulatory services, Uber, Lyft), EMS systems, pharmaceutical delivery systems, grocery and/or household item sales and delivery systems, email and telephone communication systems, social network platforms, governmental systems at the local, state, regional, and federal levels, and so on. In some embodiments, the communications operations center may include one or more operations servers operating one or more operations processors, as well as a storage system and a secure file exchange.

The storage system may include one or more HIPAA-compliant databases. In one embodiment, the storage system may include a HIPAA-compliant patient database, a HIPAA-compliant healthcare provider database, a HIPAA-compliant third-party service provider database, and a HIPAA-compliant channel database. Each of the patient, healthcare provider, third-party service provider, and channel databases may be communicatively coupled with the operations server running the operations processors, as shown in the schematic of FIG. 1, and may store any appropriate data and/or information required to implement the communication tool.

Generally speaking, the databases may include any suitable type of application or data structure that may be configured as a data repository. For example, the databases may be configured as relational databases that include one or more tables of columns and rows that may be searched or queried according to a query language, such as a version of Structured Query Language (SQL). Alternatively, the databases may be configured as structured data stores that include data records formatted according to a markup language, such as a version of eXtensible Markup Language (XML). In other embodiments, the databases may be implemented using arbitrarily or minimally structured data files managed and accessible through any suitable type of application or the databases may be non-relational or NoSQL databases. The HIPAA-compliant databases may also be decentralized and implemented via distributed ledger blockchain technology.

The patient, healthcare provider, third-party service provider, and channel databases, as well as the operations server and processors may be co-located at the communications operations center. In some embodiments, these components, or sub-components thereof, may be combined or distributed in any appropriate manner across multiple locations and/or distributed computing platforms.

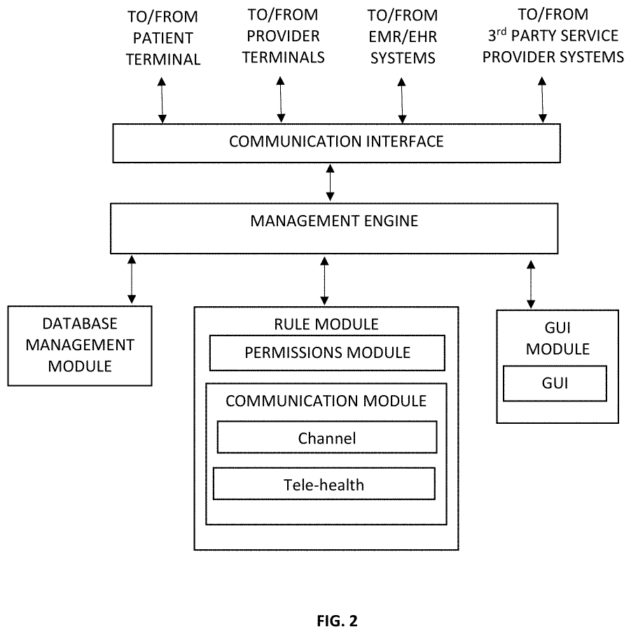

FIG. 2 provides a functional block diagram depicting illustrative communications software according to some embodiments. In various implementations, such software may be executed by the operating processors of the communications operations center. As shown, a management engine may be coupled to a communication interface, a database management module, a rule module, and a graphical user interface (GUI) module. In turn, the communication interface may be communicatively coupled with the user terminals operating at the patient and the healthcare group(s). The communication interface may also connect with one or more external systems or applications such as the EMR/EHR systems and the third-party service provider systems in secure network communication with the server.

The management engine may be configured to perform a variety of operations related to implementing the communication tool for facilitating asynchronous and synchronous communication between the patient and the various providers of the healthcare provider groups within the patient's care team, as detailed below.

The communication interface may enable the communications software to securely exchange information with other systems and/or system components via the network, including the patient and provider user terminals, as well as the one or more EMR/EHR systems and/or the external third-party service provider applications or systems. In some embodiments, the communication interface may be configured to transmit and/or receive information using secure socket layer (SSL) encryption. Additionally or alternatively, other connections may also be used, such as, for example, XML file transmission utilizing file transfer protocol (FTP), hypertext transfer protocol (HTTP) POST transactions, or other data transmission protocols. The communication interface may further include any of a variety of standardized application programming interfaces (APIs) configured to allow different software programs to communicate (e.g., to request services and respond to such requests) in an autonomous, web-based, and/or platform-independent manner. For example, the user terminal operated by the patient may choose or be operated to expose certain data (e.g., medical questions, prescription information, patient preferences, assessment information, diagnosis information) via a HIPAA-compliant, secured web interface. The communication interface may then access the exposed data and/or functions via the appropriate API(s).

The database management module may include any suitable database management system (DBMS) or application configured to manage the creation, maintenance, and use of the stored data/databases of FIG. 1. The rule module may include one or more sets of rules, in any suitable format, that provide a framework for the administration of the communication tool, as described in further detail below.

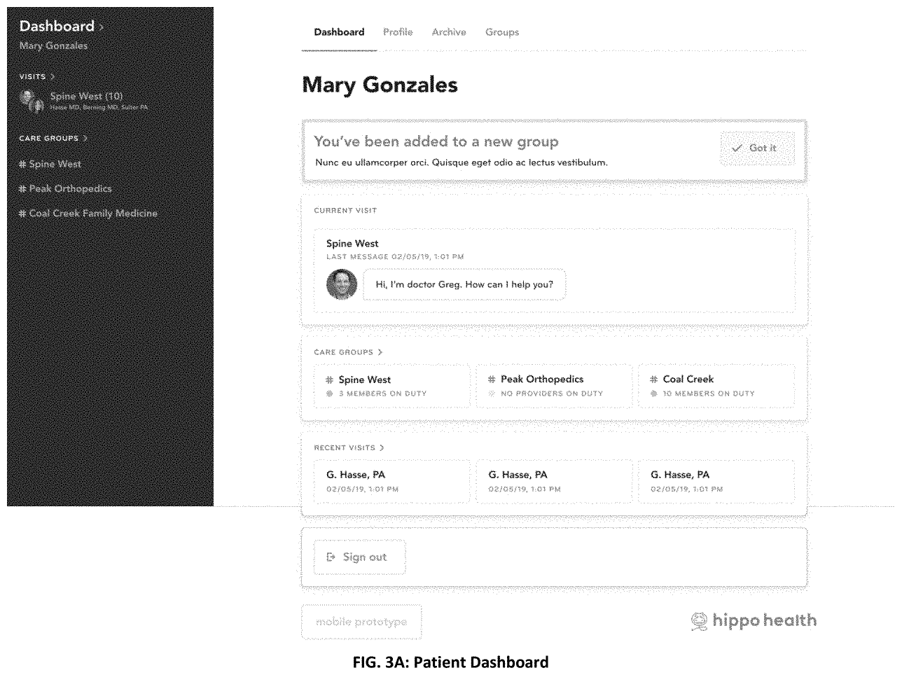

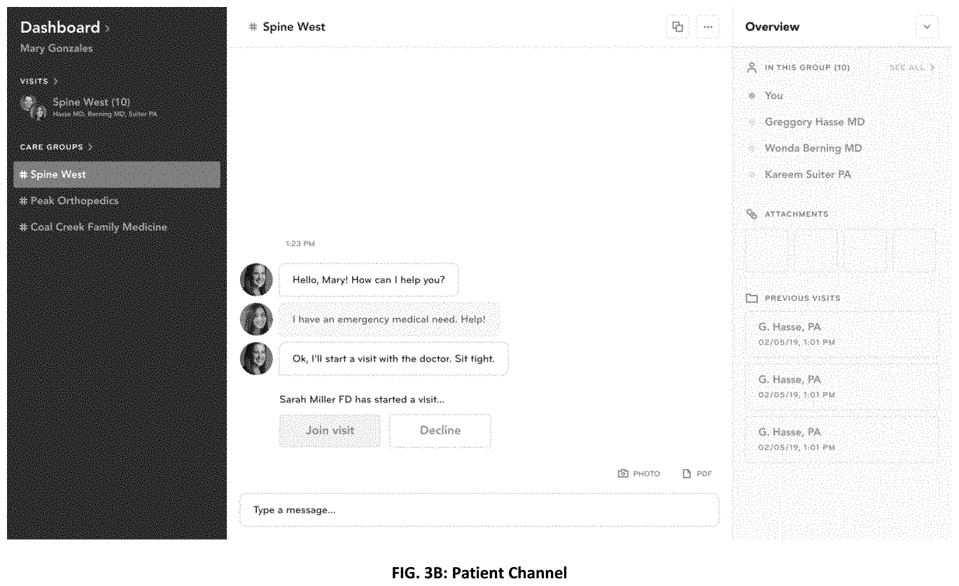

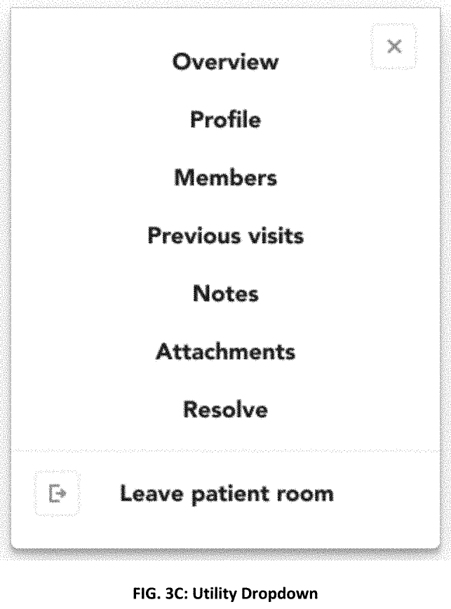

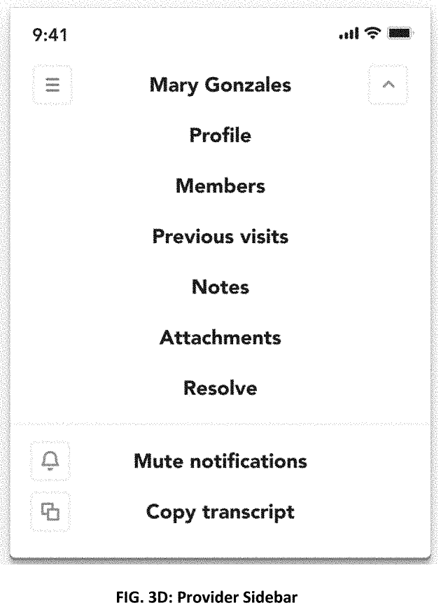

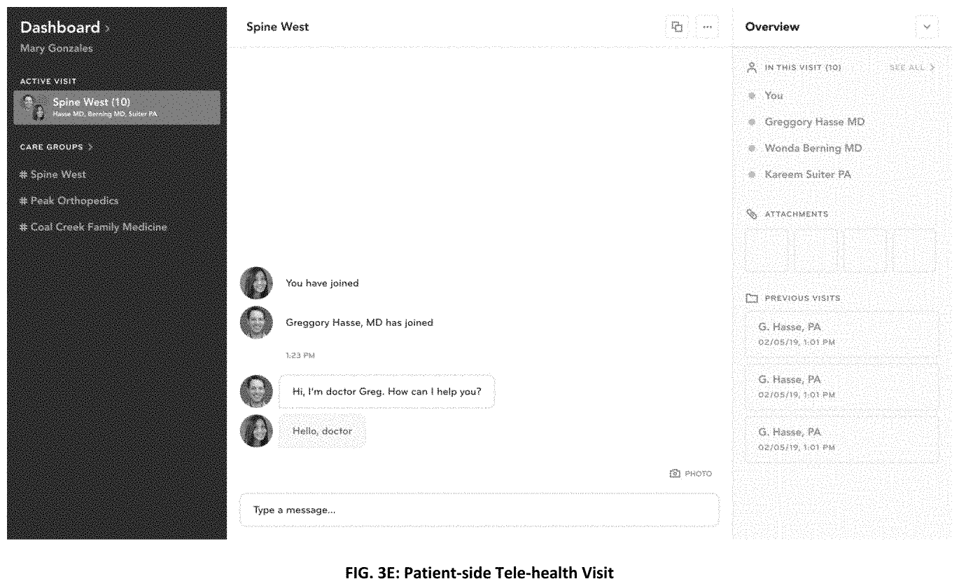

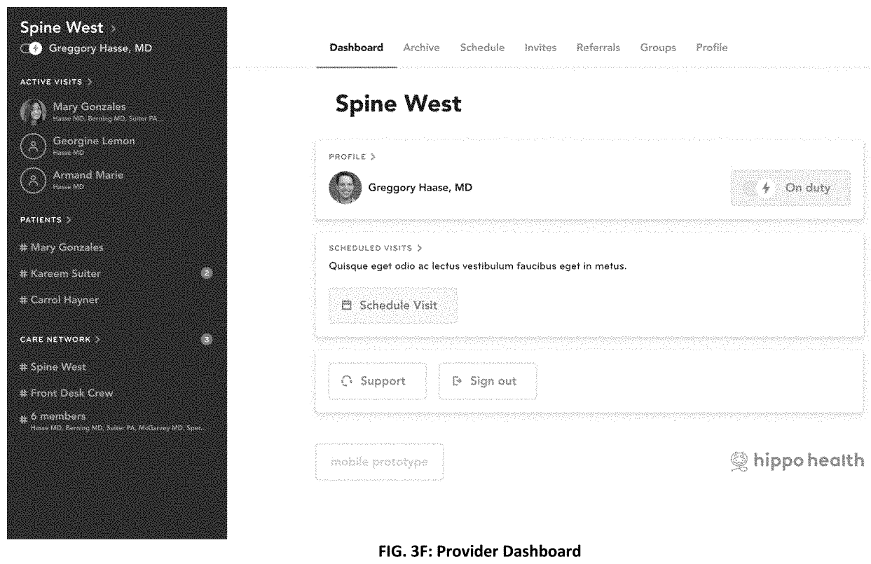

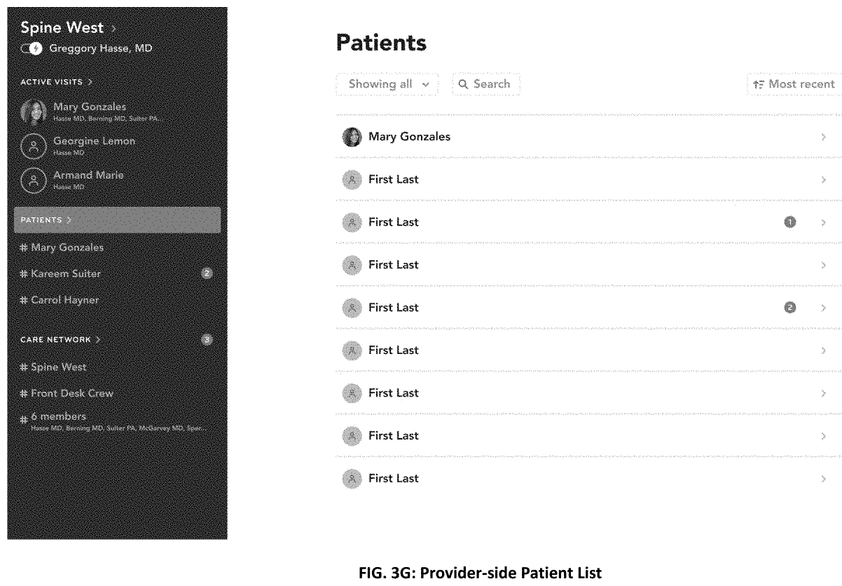

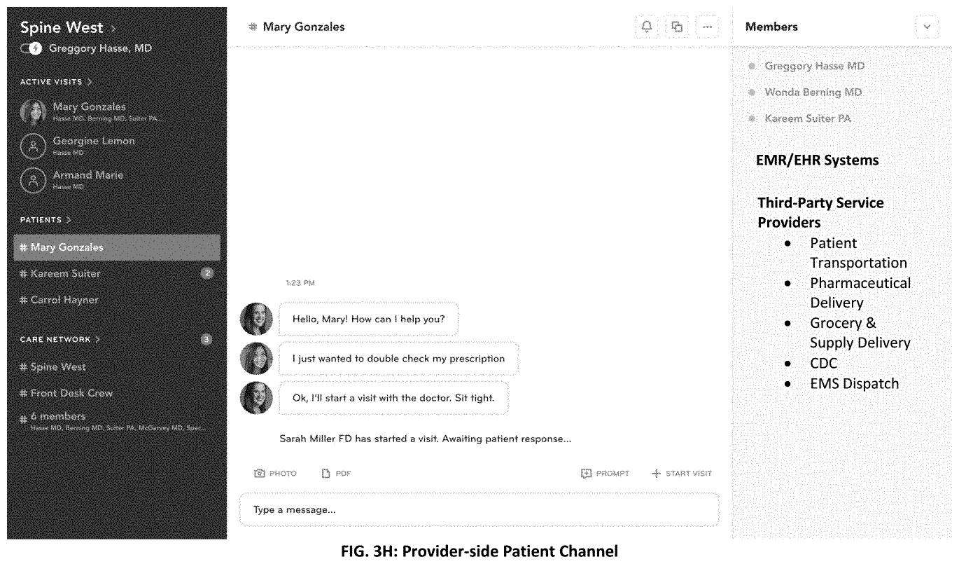

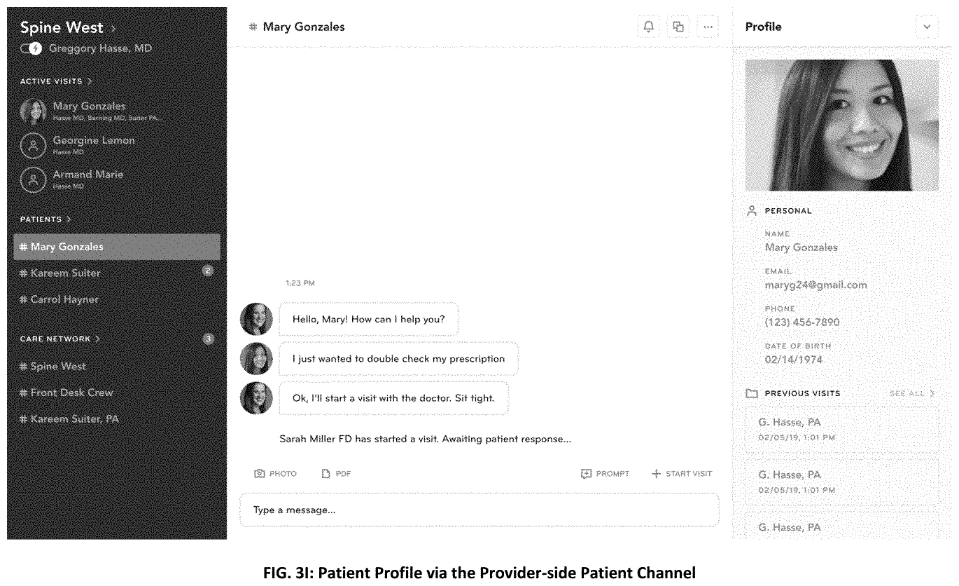

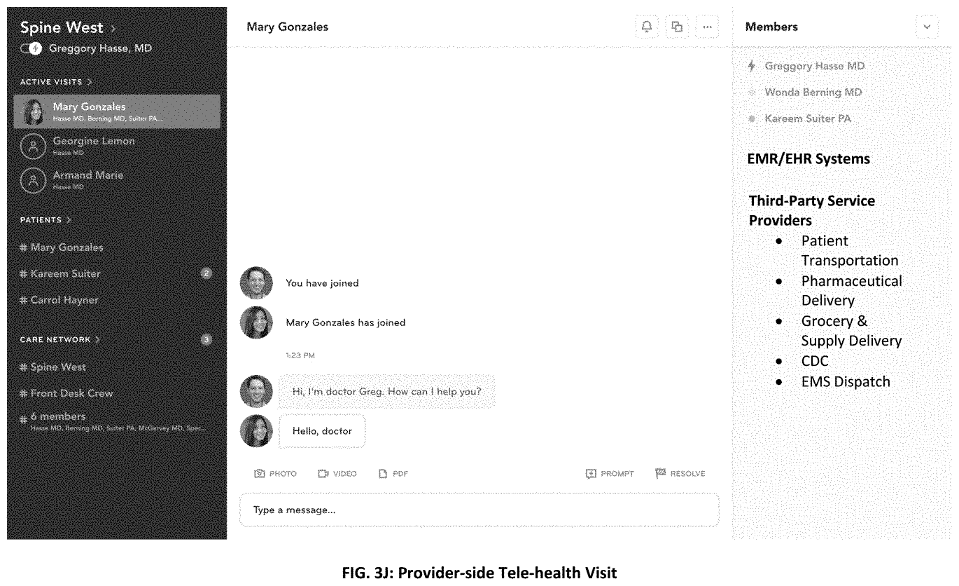

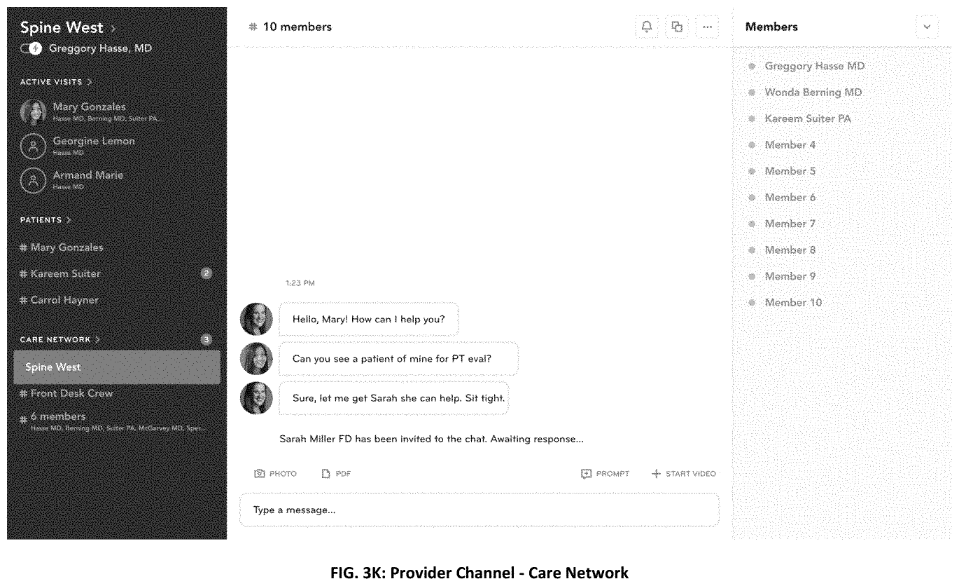

For example, the rule module may include a permissions module that instructs connectivity and privacy across the various patient and provider users, the EMR/EHR systems, the third-party service provider systems, and any additional external applications, including permissions defined at the provider group, individual user, and/or role levels to determine system access to various system software modules and content. The rule module may also include a communication module including instructions pertaining to the implementation of the channel-based communication tool, including a patient-channel module and a tele-health visit module to implement a variety of synchronous and asynchronous communication functionalities between the patient and providers, including the transition into a tele-health session, as described in further detail below in relation to the exemplary preconfigured, interactive screens of FIGS. 3A-3K.

The GUI module may be configured to provide, for example, via a web-based user interface (WUI) that implements JAVA®, AJAX®, ADOBE FLEX®, MICROSOFT.NET® or the like, a native mobile application interface such as iOS, Android, or similar technologies, for real-time user control from any appropriate user terminal. In other cases, the GUI module may implement a command line interface, an application interface, or another suitable interface using non-web-based technologies.

In various embodiments, a GUI may be displayed to system users via the GUI module of FIG. 2. The GUI may be operable to display information and/or receive commands from the user(s). In various implementations, the GUI may be displayed, via a number of appropriate preconfigured and interactive screens displayed at the user terminals operating at the patient and the healthcare provider group via the communication interface and the network. Numerous exemplary preconfigured and interactive screens demonstrating functionality examples of the communication tool are discussed below in relation to FIGS. 3A-3K.

A browser or application window displayed on any of the user terminals may be configured to display text content, image content, input features, navigable links, etc. of the interactive and preconfigured screens of the GUI. Each preconfigured screen may include any appropriate type of content in various combinations, and the screen(s) displayed to the users may be specific to the viewing platform. For example, the screen(s) presented at the terminal operated by a healthcare provider of the provider group may differ from the screen(s) shown at the terminal operated by the patient, depending on a variety of factors including, for example, the type of information to be collected or transmitted, security concerns, user permissions, the type/screen size of terminal being operated, and so on.

The content of the interactive and preconfigured GUI screens may be interspersed or combined in any suitable fashion according to the capabilities of the browser and the language used to implement the GUI by the GUI module, and may be displayed in any suitable area of the browser or application window at the user terminal. In some embodiments, the window may be generated and managed by a web browser such as, for example, MICROSOFT EXPLORER®, FIREFOX®, SAFARI®, CHROME®, etc., implemented from the communications operations center.

In various embodiments, the modules shown in FIG. 2 may represent sets of software routines, logic functions, and/or data structures that are configured to perform specified operations. Although these modules are shown as distinct logical blocks, in other embodiments, at least some of the functionality provided by these modules may be combined into fewer blocks or parceled into additional blocks. Conversely, any given one of the modules may be implemented such that its functionality is divided among two or more logical blocks. Moreover, although shown with a particular configuration, in other embodiments these various modules may be rearranged in other suitable ways.

The various systems and components shown in FIGS. 1-2 may allow the implementation of the communication tool, including the execution of methods relating to tracking the patient's care team, or the healthcare groups on the platform to which the patient belongs, generating a continuously persisting channel for the patient with each of the patient's care groups, facilitating asynchronous text-based chat on each channel between the patient and the providers of the care group, uploading images and/or files for viewing by the providers on the channel, inviting additional providers to the channel, and facilitating the transition from asynchronous communication on the channel to a synchronous virtual tele-health session between the patient and one or more of the providers on the channel using one or more tele-health tools associated with the patient and the provider user terminals.

Turning to exemplary tool functionalities, FIGS. 3A-3K illustrate numerous exemplary preconfigured and interactive screens presented at the patient and the provider user terminals in implementing the functionality of embodiments of the communication tool disclosed herein. Specifically, FIG. 3A illustrates an exemplary patient dashboard screen. In one embodiment, the patient may be a quarantined or socially distanced patient such as a COVID-19 patient operating a patient user terminal within his or her own home. Once authenticated, the patient may view a dashboard that displays a list of the healthcare groups on the platform (e.g., the care groups that are members of the larger care network platform) to which the patient belongs. This list also displays in the primary left sidebar navigation. By clicking on a care group, such as, for example, Spine West, on either the dashboard or the sidebar, the patient is brought to the patient's dedicated channel for that care group.

FIG. 3B illustrates an exemplary patient-channel screen. In this embodiment, patient channels are generated for each patient of each care group and then persist continuously. The patient channel consists of asynchronous text-based chat between the patient and multiple members or providers of the care group. The patient may select from a variety of tools to add data or files to the chat, such as uploading an image or a PDF file. A contextual right sidebar navigation presents additional information about the channel chat and the care group, including, for example, the member providers presently in the chat and their status, past tele-health visits between the patient and providers of the care group, and previously uploaded attachments. The patient may see a “join visit” prompt within the chat transcript if a provider in the channel chat starts a tele-health visit. Upon clicking to join the visit, the patient is brought to a tele-health visit.

FIGS. 3C-3D illustrate an exemplary utility dropdown menu and a provider sidebar presenting additional tools/functions available to the patient and the providers within the patient channel including, for example, options to copy the channel transcript, send the transcript to EHR, mute notifications from the channel, invite others to the channel, and/or leave the channel.

FIG. 3E illustrates an exemplary tele-health visit screen from the patient-side of the patient channel, comprising an asynchronous text-based chat, as well as an option for synchronous video chat. Tele-health visits may be temporary with discrete start and end times, managed by the provider.

FIG. 3F illustrates an exemplary provider dashboard screen. Once authenticated, providers may view a primary left sidebar navigation that lists patient channels for the provider group by patient name. By clicking on a patient name, such as, for example, Mary Gonzales, the provider is brought to the patient channel for that healthcare provider group. For instance, by clicking on the patient name, a provider with the Spine West provider group is brought to the Mary Gonzales channel for the Spine West provider group. The patient-channel list is displayed by most recent activity and also includes visual notifications such as, for example, a visual queue that displays relevant items in the sidebar list, including a count of unread messages or a notification to providers that a given patient channel has activity that requires attention.

FIG. 3G illustrates an exemplary patient-list screen for a provider. Upon clicking the section header “Patients” within the primary left sidebar navigation of the provider dashboard screen of FIG. 3F, the provider may be brought to a list of all patient channels by patient name, as shown in FIG. 3G. The list may default to list patient channels by most recent activity, and providers may use filter, sort, and search tools to find particular patient channels within the list. Similar to the list in the primary sidebar navigation, a visual queue may indicate a patient channel with activity that requires attention.

FIG. 3H illustrates an exemplary screen presented to the provider after entering the patient channel for that patient-care provider group (i.e., the “provider-side patient channel”). Upon clicking a patient channel name in the left sidebar navigation of the provider dashboard or from the patient list, the provider is brought to the patient channel for that patient-care provider group. In this embodiment, providers have all of the same tools as patients within a patient channel, including options to add data or files to the chat and view additional information about the chat. In addition, and unique to providers, providers may click to start or initiate a tele-health visit with the patient, which, in turn, displays a prompt to join the tele-health visit in the chat transcript displayed to the patient, as shown in FIG. 3B. Prior to, during, or after a tele-health visit with the patient, the healthcare provider may select from a variety of options to engage with EMR/EHR systems and/or one or more third-party service provider systems, as selected from the provider-side patient channel, shown in FIG. 3H.

FIG. 3I illustrates an exemplary patient profile screen via a sidebar accessible from the provider-side patient channel screen. In this embodiment, a provider may view information about the patient within the contextual right sidebar navigation within the provider-side patient channel. The profile may include, for example, contact information, medical history, and previous visits.

FIG. 3J illustrates an exemplary tele-health visit screen from the provider-side of the patient channel. As discussed above, a provider may initiate a tele-health visit by clicking on the “start visit” button in the provider-side patient channel, shown in FIG. 3H. Doing so displays a “join visit” button in the patient-side chat transcript, as shown in FIG. 3B. When the patient clicks the “join visit” button, the provider is linked into the patient tele-health visit, as shown in FIG. 3J. As discussed above, at anytime during the tele-health visit, the provider may link to one or more external third-party service providers to provide the patient with transportation, pharmaceutical delivery, food or supply delivery, emergency services, notification services, and/or reporting and/or support via one or more government organizations such as the CDC. In some embodiments, the patient may access these options, or any appropriate subset of or additions to these options, through the patient-side of the patient channel.

FIG. 3K illustrates an exemplary provider channel screen, similar to a patient channel screen. That is, there is a provider channel for each healthcare group on the communication tool/platform. Provider channels can be found listed in the primary left sidebar navigation in the “care network” section. As well, clicking on the “care network” section header takes the provider to a directory that lists all of the provider channels. When a provider clicks on a provider channel name, the provider is brought to the provider channel for that provider-care provider group. The provider can then interact with the care provider group similar as to how a patient interacts with a care provider group through the patient channel.

In addition to communication between patients and healthcare professionals and healthcare professionals with other healthcare professionals, patients and healthcare professionals may communicate via the patient and the provider channels with any relevant providers of the care group, including, for example, billing personnel, scheduling personnel, administrative personnel, and the like.

Using embodiments of the communication tool discussed above, patients are able to communicate with multiple healthcare providers working with each of the provider groups they belong to, through a single patient channel for each care group that tracks and organizes the information provided from several different people, at different times, from different user terminals. This ability to communicate asynchronously allows for more effective communication and, therefore, care improvements from the provider(s) to the patient, as well as for passive monitoring, workflow management ensuring the most relevant provider is brought into the channel, triage of inbound patient inquiries, provision of external services outside the offerings of, but requested by, the healthcare provider(s), and ultimately, for seamless transition to synchronous tele-health visits when necessary and appropriate. Providers may also communicate more effectively and in a streamlined manner with their patients, colleagues, and other provider-group personnel. Ultimately, embodiments of the communication tool and associated methods discussed herein optimize personalized care for each patient to improve patient engagement, treatment adherence, the patient experience, the care team experience, health outcomes, and the cost of care.

Embodiments of the communication tool disclosed herein provide an ideal platform to deliver telemedicine services to pandemic patients who are in quarantine or who are socially distanced or isolated, including patients at risk for or infected with COVID-19. Using embodiments of the communication tool discussed above in relation to FIGS. 1-3, healthcare providers, including EMS providers and local emergency response agencies, are able to respond to and treat COVID-19 patients via the platform from locations remote to the patients' physical locations and/or homes. Via tele-health visits, healthcare providers are able to assess the need to either treat the patient in place or transport the patient, if needed, without direct contact, eliminating the risk of spreading the virus to critical healthcare providers, thereby keeping the healthcare providers healthy and in the workforce and minimizing the use of rapidly depleted personal protective equipment (PPE).

Depending on the care the patient requires, healthcare providers are able to provide screening and ongoing monitoring of symptoms, to triage patients located at home, and able to dispatch EMS if and when necessary. In some embodiments, the communication tool enables communication between EMS teams providing on-site assessment, treatment, and/or stabilization of COVID-19 patients at home and appropriate physicians selected from the patient's care team, all through the patient's channel on the communication tool. Healthcare providers are additionally able to arrange for pharmaceutical, grocery, and/or supply delivery to the home in instances where it remains appropriate for the patient to shelter in place or to arrange for non-emergency transportation to alternate destination sites for clinical care.

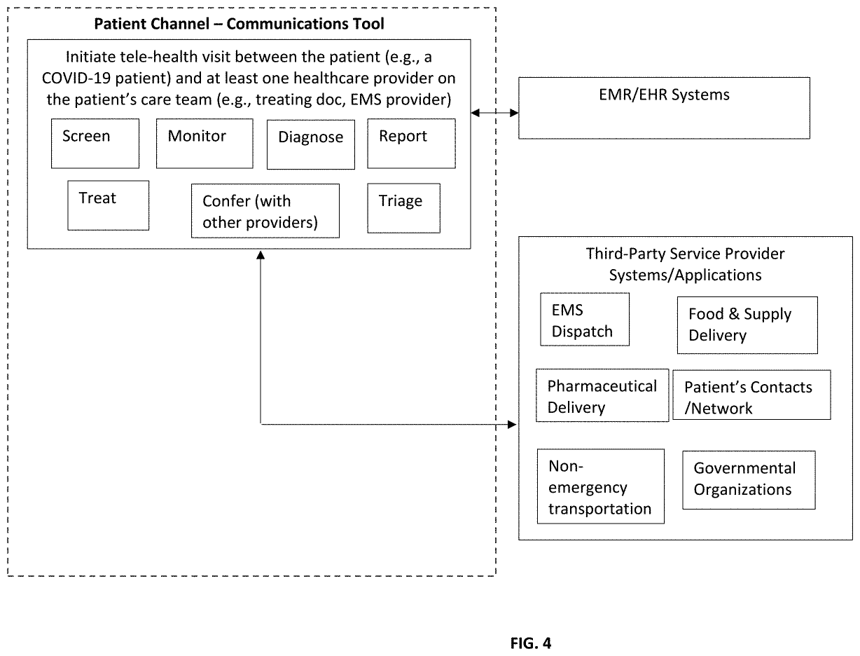

FIG. 4 provides a flowchart depicting an exemplary method of treating a quarantined pandemic patient (e.g., a COVID-19 patient) using the communication tool discussed above. In this embodiment, the method begins with an initiation of a tele-health visit, via the patient channel, between the patient and at least one healthcare provider on the patient's care team. This healthcare provider may be the patient's usual treating healthcare provider or physician or a member of an EMS team or emergency response agency provider.

During the tele-health visit, the healthcare provider may provide a variety of healthcare services related to screening, monitoring, diagnosis, treatment, and triage, if necessary, using the patient and provider user terminals and the associated tele-health tools. Before, during, or after the tele-health visit, the healthcare provider may opt to initiate communication with one or more EMR/EHR systems to request patient information and/or to report it. The healthcare provider may also initiate communication with one or more third-party service provider systems or applications to provide services such as, for example, arranging for EMS dispatch, requesting food and supply delivery to the patient's location, requesting pharmaceutical delivery to the patient's location, notifying the patient's family and/or friends via telephone, email, and/or social media platforms, requesting non-emergency transportation through a cab, ride-share service, or public transport to an alternative destination site for clinical treatment, and reporting to appropriate local, state, regional, or federal organizations.

In one embodiment, if EMS services are dispatched to the patient's location, the EMS team may participate in the tele-health visit, or initiate a new visit, upon arrival in an effort to assist the healthcare provider communicating through the tool in providing real-time patient assessment and treatment.

Using embodiments of the communication tool and the methods discussed above, healthcare providers may work with local, regional, and state organizations to provide a mobile platform to deliver telemedicine services to COVID-19 patients. EMS and local emergency response agencies may respond to and treat patients from outside their homes via the platform, and may assess the need to treat in place and transport if needed without direct contact. The system allows critical providers to minimize exposure to COVID-19, keeping them healthy and in the workforce, and minimizing the use of the depleting personal protective equipment (PPE).

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A healthcare communication tool for synchronous and asynchronous communication between a patient and a plurality of healthcare providers of a healthcare provider group, comprising:
a communications operations server operating one or more processors in communication with a patient database, a healthcare provider database, a third-party service provider database, and a channel database; and
a management engine running on the communications operations server, the management engine executing a database management module, a rule module, and a graphical user interface (GUI) module configured to display a GUI having a plurality of preconfigured, interactive screens to users operating at least one healthcare provider terminal operatively coupled with a plurality of provider tele-health tools and a patient terminal operatively coupled with a plurality of patient tele-health tools, the management engine configured for:
initiating, via a patient channel dedicated to the patient, a tele-health visit between the patient and at least one of the plurality of the healthcare providers;
asynchronously exchanging, via the patient channel and using the plurality of the patient and the provider tele-health tools, information relating to one or more of patient screening, monitoring, diagnosis, treatment, and triage;
reporting to one or more electronic medical record (EMR) systems, the information relating to the one or more of the patient screening, monitoring, diagnosis, treatment, and triage; and
requesting, from one or more third-party service provider systems, one or more of an emergency medical services dispatch, a food delivery to the patient, a supply delivery to the patient, a pharmaceutical delivery to the patient, and a non-emergency transportation service for the patient.

2. The healthcare communication tool of claim 1, the management engine further configured for:
reporting, to one or more governmental organization systems, the information relating to the one or more of the patient screening, monitoring, diagnosis, treatment, and triage.

3. A healthcare communication tool for synchronous and asynchronous communication between a patient and a plurality of healthcare providers of a healthcare provider group, comprising:
a communications operations server operating one or more processors in communication with a patient database, a healthcare provider database, a third-party service provider database, and a channel database; and
a management engine running on the communications operations server, the management engine executing a database management module, a rule module, and a graphical user interface (GUI) module configured to display a GUI having a plurality of preconfigured, interactive screens to users operating at least one healthcare provider terminal operatively coupled with a plurality of provider tele-health tools and a patient terminal operatively coupled with a plurality of patient tele-health tools, the management engine:
receiving, via a patient channel dedicated to the patient and the plurality of the healthcare providers of the healthcare provider group, a healthcare request from the patient operating the patient terminal;
displaying, within the patient channel and upon one of the preconfigured, interactive screens of the GUI at the at least one healthcare provider terminal and the patient terminal, the healthcare request;
receiving, via the patient channel, a healthcare response from at least one of the plurality of the healthcare providers operating the at least one healthcare provider terminal; and
displaying, within the patient channel and upon another one of the preconfigured, interactive screens of the GUI at the at least one healthcare provider terminal and the patient terminal, the healthcare response.

4. The healthcare communication tool of claim 3, the management engine further:
asynchronously receiving, via the patient channel, additional healthcare responses from the patient operating the patient terminal and the at least one of the plurality of the healthcare providers operating the at least one healthcare provider terminal and displaying, within the patient channel and upon another one of the preconfigured, interactive screens of the GUI at the at least one healthcare provider terminal and the patient terminal, the additional healthcare responses to form a continuous patient-channel transcript within the patient channel.

5. The healthcare communication tool of claim 4, the management engine further:
receiving, via the patient channel, a tele-health visit initiation from the at least one of the plurality of the healthcare providers operating the at least one healthcare provider terminal;
receiving, via the patient channel, a tele-health visit acceptance from the patient operating the patient terminal; and
initiating, via the patient channel and upon another one of the preconfigured, interactive screens of the GUI at the at least one healthcare provider terminal and the patient terminal, a virtual tele-health visit.
